(19) United States
(12) Patent Application Publication
(10) Pub. No.: US 2013/0184660 A1
(43) Pub. Date: Jul. 18, 2013

(54) SELF-SEALING CATHETERS

(71) Applicant: Walkill Concepts, Inc., Los Altos, CA (US)

(72) Inventors: Gerald F. Swiss, Rancho Sante Fe, CA (US); Stefan Schwabe, Palmetto Bay, FL (US); Robert M. Moriarty, Michiana Shores, IN (US)

(73) Assignee: WALKILL CONCEPTS, INC., Los Altos, CA (US)

(21) Appl. No.: 13/720,682

(22) Filed: Dec. 19, 2012

Related U.S. Application Data
(60) Provisional application No. 61/578,627, filed on Dec. 21, 2011.
(60) Provisional application No. 61/668,955, filed on Jul. 6, 2012.

(51) Int. Cl. A61B 17/00; A61M 25/00

(52) U.S. Cl. CPC A61B 17/0057; A61M 25/0084; A61M 25/00
USPC 604/272; 604/264

(57) ABSTRACT

Provided are catheters useful for penetrating the vascular wall and delivering medicament as necessary to tissue proximate the vasculature. Such catheters are particularly useful in delivering site specific medicaments to the tissue which can be damaged by hemorrhagic stroke, ischemia, and the like. Methods of using such catheters are also disclosed.

### CROSS-REFERENCE TO RELATED APPLICATIONS

This application claims the benefit under 35 U.S.C. §119(e) of U.S. Provisional Application Nos. 61/578,627, filed Dec. 21, 2011, and 61/668,955, filed Jul. 6, 2012, the contents of each of which are hereby incorporated by reference in their entirety into the present disclosure.

### FIELD OF THE DISCLOSURE

Disclosed are self-sealing catheters useful for penetrating a vascular wall and sealing the wall after penetration. Such catheters are particularly useful in delivering site specific medicaments to extravascular tissue proximate to the penetration site. Such sites include tissue adjacent to the vasculature which is susceptible to restenosis or tissue which can be damaged as a result of a hemorrhagic stroke, ischemia, and the like. Methods of using such catheters are also disclosed.

### STATE OF THE ART

Vascular disease often leads to catastrophic results including rupture or blockage of a blood vessel. One instance of vascular disease is an aneurysm which forms from weakened vasculature. When located in the brain, aneurysmal rupture is referred to as a hemorrhagic stroke and such strokes account for about 20 percent of all strokes. (avm.ucsf.edu/patient_info/WhatIsAStroke/). These strokes are very difficult to treat as cranial ischemia is accompanied by release of blood into the cranium. The so released blood causes inflammation which, in combination with the ischemia, lends itself to high levels of nerve damage which, in many cases, leads to patient morbidity.

Another instance of vascular disease is arterial plaque buildup which, when combined with platelet aggregation or plaque fragmentation, results in complete vascular blockage which correlates to an ischemic attack. When the ischemia is in the coronary system, the common result is a cardiac arrest (heart attack). In such cases, an immediate concern after such an ischemic attack is the subsequent inflammation occurring in the damaged tissue immediately following the attack. Treatment of such inflammation is often more critical to survival than the initial ischemic insult and occurs within hours after the initial insult. The immediate onset of inflammation contra-indicates against the use of systemic anti-inflammatory. In the above cases, prompt treatment of the tissue minimizes damage such as loss of brain function, coronary tissue death or restenosis.

Still another vascular issue occurs when the arterial plaque is diagnosed prior to an ischemic event. In such cases, balloon angioplasty coupled with stent placement is one conventional therapy. A common side effect of this procedure is restenosis due in part to inflammation at the site of the angioplasty procedure arising from the damage caused to the vascular endothelium by the angioplasty. Current treatment to inhibit restenosis includes a drug eluting stent wherein the drug is slowly release. While effective in some cases, such stents are wire meshes which do not cover the entire diseased vasculature nor are they completely effective. Moreover, as the stent is placed on the vascular wall which is a non-static environment with blood continuously flowing, any drug that is released is subject to immediate transport away from the site of potential restenosis.

In view of the above, there is an ongoing need to effectively deliver a drug to tissue adjacent to diseased vasculature, preferably on the outside of the vascular wall. One of the obvious difficulties in doing so is the need to penetrate the vascular endothelium without causing unnecessary bleeding or vascular rupture.

### SUMMARY OF THE INVENTION

This disclosure, in some embodiments, is directed to a self-sealing catheter device capable of puncturing the vascular wall. In one embodiment, such catheters are capable of delivering medicament directly to a tissue which is at risk of damage or further damage. The catheter comprises a puncture device having a puncture tip which is capable of traversing the vascular wall. The catheter further provides for means to self-seal the wound at the site of the puncture so that bleeding through the puncture site is minimized. Preferably, the catheter is capable of delivering a medicament to the tissue after puncturing the vascular wall.

Accordingly, in one embodiment, there is provided a self-sealing catheter comprising proximal and distal ends and having at least one lumen traversing from the proximal to the distal ends wherein the lumen comprises a puncture device slideably engageable within the lumen wherein said puncture device comprises a self-sealing puncture tip at its distal end said tip being capable of penetrating the vascular wall and further wherein the puncture tip is optionally capable of delivering a medicament to the site punctured by the tip. In one preferred embodiment, the puncture tip is capable of sealing the puncture wound by forming a layer proximate the vascular wall at the site of the wound which seals the wound. In one preferred embodiment, the layer is a biocompatible layer which is placed on the tissue side of the vasculature puncture site so as to minimize interference with blood flow through the vasculature.

In another embodiment, there is provided a catheter comprising proximal and distal ends and having at least two lumen traversing from the proximal to the distal ends wherein the first lumen is sized to deliver an embolic composition or device to a vascular site, such as an aneurysm to arrest/inhibit blood flow into said site, and the second lumen comprises a puncture device slideably engageable within said lumen wherein said device comprises a puncture tip at its distal end which tip is capable of penetrating the vascular wall and further wherein the puncture tip is capable of delivering a medicament to the site punctured by said tip. In this embodiment, self-sealing of the puncture site occurs by virtue of the arrest of blood flow through the embolic composition such that natural coagulation will result in sealing.

In either embodiment, the puncture tip is self-sealing. Further examples of self-sealing tips include those where the tip, after puncturing the vascular wall, can be flattened to form a surface proximate the puncture wound. In such an embodiment, the tip goes from an elongated portion along the Z-axis to a fairly flat portion on the Z-axis but extending on the X and Y axis so as to seal the puncture site. Alternatively, the tip can include an optional extendible collar proximate the tip portion of the catheter in such a manner that the collar opens upon penetration of the vasculature so as to form a flattened surface proximate the puncture wound which seals the puncture site. In a preferred embodiment, the collar is made of a biocompatible expandable material which contains one or more medicaments.

Another embodiment provides a catheter comprising a lumen traversing the length of the catheter which lumen contains a puncture device which is slideably engageable along the length of said lumen, said puncture device comprising a head portion, a neck portion, a stem portion wherein said head portion is at the distal end of the puncture device and comprises a base which narrows at its distal end to form a puncture tip, the base having a cross section that is sized such that the head portion moves along within the lumen and is capable of being recessed within the lumen of the catheter until used, the neck portion comprising a depression as defined by the width of the base of the head portion and the width of the stem portion of the puncture device, the depression holding an expandable material which, when in the lumen of the catheter wall, is prevented from expansion and when released from the catheter wall, expands to form a collar.

In one preferred embodiment, the puncture device contains at least one defined detachment point so as to separate the distal portion of the catheter from the proximal portion.

In one preferred embodiment, the puncture device contains an internal lumen running the length of the device and capable of delivering medicament into and through the puncture tip. In another preferred embodiment, the head portion of the puncture tip contains one or more pores in communication with a lumen traversing the puncture device. These pores form micro-channels throughout which, for example, permit the delivery of a medicament through the catheter into the tissue once the tip punctures the vascular wall. Alternatively, if the head portion comprises a biodegradable material such as collagen, introduction of collagenase can be included as a final step prior to detachment of the distal portion of the puncture device so as to facilitate rapid degradation of the head portion. In such a case, the micro-channels allow the collagenase solution to permeate through the head portion so as to facilitate degradation.

In another embodiment, the puncture tip comprises two components, an outer tip portion and an inner spherical or rounded delivery unit which is contained within the outer tip. The outer tip portion is releaseably engaged from inner delivery unit so as to expose the spherical or rounded delivery unit for delivering medicament to the tissue. For example, the outer tip can be a thin shell of collagen or other biodegradable material which is retained in place by a limited amount of cohesiveness with the remainder of the puncture device. High pressure can be applied to the outer tip by an aqueous solution being pushed through the lumen of the puncture device until the tip is dislodged. As before, the aqueous solution can contain an enzyme to facilitate degradation of the tip.

Once the puncture tip is dislodged, the underlying spherical or rounded delivery unit will act to deliver the medicament. In such cases, the delivery unit is designed to not contain edges so it can be manipulated after puncture to direct the delivery of medicament to targeted areas without tissue tearing.

In one embodiment, the puncture tip is made of a biodegradable material. In another embodiment, the collar is optionally made of a biodegradable material. In yet another embodiment, the remainder of the head portion is made of a biodegradable material.

In another embodiment, the expandable collar or expandable material is optionally bound to the vertical portion of the neck of the catheter so as to form a tight seal at the vascular wall. For example, the portion of the collar can be bound to the surface of the neck of the puncture unit by an adhesive, heat melting and the like so that the collar becomes an integral part of the neck. Still further, the expandable collar can be formed to expand primarily in the horizontal plane so as to cover a large area over the puncture site.

In yet another embodiment, the tip of the catheter comprises a biocompatible polymer that allows the lumen to be readily closed by contact with a biocompatible solvent such as DMSO, ethanol, and the like. Upon contact, the biocompatible material partially dissolves into the lumen thereby blocking the lumen. The polymer material can contain a contrast agent integrated therein such that the clinician can monitor the closing of the lumen.

### BRIEF DESCRIPTION OF THE DRAWINGS

FIG. 1A-F are side views of one embodiment of the catheter device, at different steps of a process to penetrate a vascular site, of the present disclosure.

FIG. 2A-D are side views of another embodiment of the catheter device of the present disclosure.

FIG. 3 shows one embodiment of the catheter device of the present disclosure viewed from the side when the device has penetrated the vascular wall so as to deliver a medicament to a vascular site.

FIG. 4A-F show the side view of another embodiment of the catheter device and the process of using the device to penetrate and seal a vascular wall.

FIG. 5 illustrates an interlocking mechanism that releaseably engages different portions of a catheter.

### DETAILED DESCRIPTION

Before the compositions and methods are described, it is to be understood that the disclosure is not limited to the particular methodologies, protocols, and devices described, as these may vary. It is also to be understood that the terminology used herein is intended to describe particular embodiments of the present disclosure, and is in no way intended to limit the scope of the present disclosure as set forth in the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

When a numerical designation is preceded by the term “about”, it varies by (+) or (−) 10%, 5% or 1%. When “about” is used before an amount, for example, in mg, it indicates that the weight value may vary (+) or (−) 10%, 5% or 1%.

### Definitions

In accordance with the present disclosure and as used herein, the following terms are defined with the following meanings, unless explicitly stated otherwise.

As used in the specification and claims, the singular form “a”, “an” and “the” include plural references unless the context clearly dictates otherwise. For example, the term “a cell” includes a plurality of cells, including mixtures thereof

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed disclosure. “Consisting of” shall mean excluding more than trace amount of other ingredients and substantial method steps recited. Embodiments defined by each of these transition terms are within the scope of this disclosure.

The term “self-sealing” as used herein means that the puncture of the vasculature formed by the puncture device will limit the amount of bleeding through the puncture in a manner that when combined with natural thrombosis and/or an embolic composition will result in sealing the wound with limited amount or no blood traversing through the puncture wound. Preferably, sealing will occur within about 5 minutes of puncture and more preferably within about a minute of puncture.

The term “slideably engageable” means that the puncture device can readily move both in a forward and reverse direction within the lumen of the catheter.

### Self-Sealing Catheter Devices

The present disclosure provides catheters having one or more lumen wherein within at least one of said lumen there is a slideably engageable puncture device that can traverse the lumen and, when appropriate, puncture a vascular or arterial wall (e.g., from the intra-arterial to the outer arterial direction). In one embodiment, the puncture is from the intra-arterial to the outer arterial direction so as to deliver a medicament to the tissue adjacent to the punctured vascular site or to remove material from that tissue. The catheters of this disclosure provide for self-sealing of the puncture wound so as to inhibit bleeding across the vascular wall.

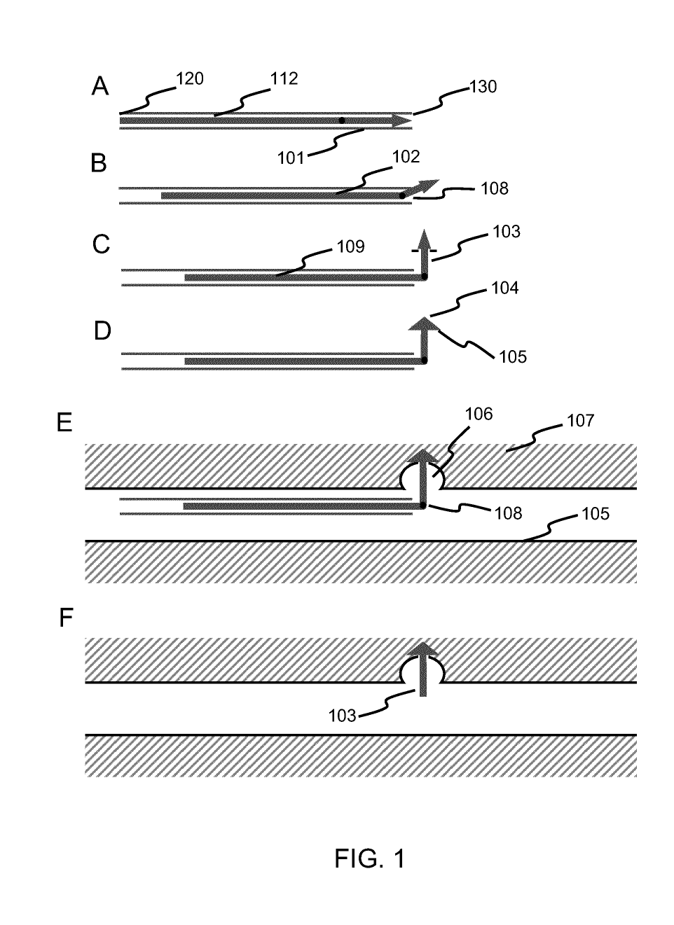

With reference to FIG. 1, one embodiment of the present disclosure as shown in FIG. 1A through 1D provides a catheter (not shown) having an internal wall 101 which defines a lumen 112 having a proximal end 120 and a distal end 130 which is open. The lumen 112 contains a puncture device 102 slideably engageable therein. The puncture device has a catheter tip 104 and a neck portion 103 which connects the tip 104 to the remaining stem 109 of the puncture device 102. Puncture tip (104) is capable of puncturing the interior vascular wall of an artery.

The distal end of the puncture device 102 as well as the catheter are made of well known flexible components which permit the catheter to traverse tortuous regions of the vasculature. Examples of such catheters and components are found in, for instance, U.S. Pat. Nos. 5,704,926, 6,500,147, 6,171,294, and 5,961,510. In some aspects, the catheter or puncture device is coated with a polymer that facilitates sliding. Such polymers are known in the art. See, for instance, U.S. Pat. No. 7,060,372.

FIGS. 1B-F illustrate the flexibility of the distal portion of the puncture device. Such allows the clinician to align the tip of the catheter and the puncture device to the appropriate position 105 within the vasculature. Specifically, as shown in FIGS. 1B-D, the puncture device contains a head portion 103 having a tip 104 and a base 103. When the puncture device is enclosed in the lumen 112 defined by the inner catheter wall 101, the wall keeps the puncture device substantially straight. If the puncture device is extended from the catheter wall, the flexibility of the distal end of the puncture device permits the clinician to move that end independent of the catheter.

In one aspect, the base 103 of the puncture tip can be opened as shown in FIG. 1D so as to initiate a process wherein the tip is converted into a relatively flat surface which, as the puncture device is retracted, will form a sealing surface over the puncture wound. In one embodiment, the tip will stay closed when moving in the forward direction, and will expand into a flat surface when moved in the reverse direction, particularly when under pressure. Alternatively, the tip can be maintained in its rigid form by a dissolvable seal which can be dissolved by use of a compatible solvent such as DMSO, ethanol, ethyl lactate, and the like which can be injected into the tip area through the lumen traversing the puncture device after it is positioned. Once dissolved, the tip will open to a flat surface much like opening an umbrella.

In one embodiment, the puncture device itself also contains a lumen which permits the delivery of a medicament to the outside of the vasculature once the seal is dissolved. In another embodiment, the tip of the puncture device is impregnated with the medicament or a mixture of medicaments such that the medicament(s) is (are) slowly released as the tip biodegrades.

FIGS. 1E and 1F show another embodiment wherein the puncture device traverses an aneurysm such that the aneurysm does not rupture or further rupture. In this embodiment, the puncture tip is positioned immediately against the wall 105 of the aneurysm. A second catheter (not shown) delivers an embolic composition into the aneurysm such that blood flow into the aneurysm is significantly inhibited or stopped. After the second catheter is removed, the puncture tip of the catheter of this disclosure is moved to puncture the vascular wall and deliver medicament. As the embolic composition has arrested blood flow, such puncturing can be achieved with minimal or no bleeding at the site of the puncture. Once the puncture is complete, the puncture device can be substantially removed merely by applying backward force which will break the puncture device and the catheter itself at a predetermined detachment point (e.g., 108). The portion of the puncture tip remaining in the vasculature will be biodegradable so that over time, their will be no remnant of the tip remaining. Suitable embolic compositions are well known in the art and include platinum coils as well as an in vivo hardening composition such as cyanoacrylates or ONYX® embolic formulation (available from Covidien, Irvine, Calif., USA)

In one aspect, the vascular wall is intact but is punctured by the puncture tip. In another aspect, the puncture size formed in the vascular will be smaller than the collar when expanded in the tissue thereby sealing the opening on the vascular wall (FIGS. 1D and 1E).

In some aspects, the puncture device is breakable (or detachable) at a point (e.g., 108) proximate the puncture tip. In one aspect, the detachment point is on or immediately adjacent to the head portion (103). In another aspect, the detachment point is below the puncture tip. Once the puncture tip penetrates a vascular wall and seals it, the puncture device can be broken at the detachment point so that the catheter and a majority of the puncture device can be removed from the vascular site (FIG. 1F).

In some aspects, the puncture tip and/or collar comprises a medicament, which medicament can be released to the tissue once the puncture tip and optional collar are placed in the tissue behind the vascular wall. Types of medicaments suitable for delivery to the tissue are described below.

In one aspect, the puncture device, at the head portion or the portion at the distal end of the detachment point, is made of a biodegradable material. Therefore, the head portion or the portion of the puncture device distal to the detachment point can be left at the vascular site safely as it will degrade gradually in the patient's body.

“Biodegradable materials”, including those suitable for medical use, are known in the art. For instance, Ikada and Tsuji “Biodegradable polyesters for medical and ecological applications,” Macromol. Rapid Commun. 21:117-32 (2000) reviews polymers that safely degrade in vivo and thus are suitable biodegradable materials for the purpose of the present disclosure. In one aspect, a biodegradable material is selected from a polysaccharide, such as cellulose, starch, alginate, chitin (chitosan), hyaluronate, and hyaluronate; a protein such as collagen (gelatin) and albumin; a polyester such as Poly(3-hydroxyalkanoate); or a synthetic polymer such as Poly(ethylene succinate), Poly(butylene terephthalate), polyglycolide, polylactides, poly(c-carpolactone), poly(butylene terephthalate), poly(vinyl alcohol), poly(ester carbonate), polyanhydrides, polyphosphazenes, and poly(orthoesters). In a particular aspect, the biodegradable material is collagen.

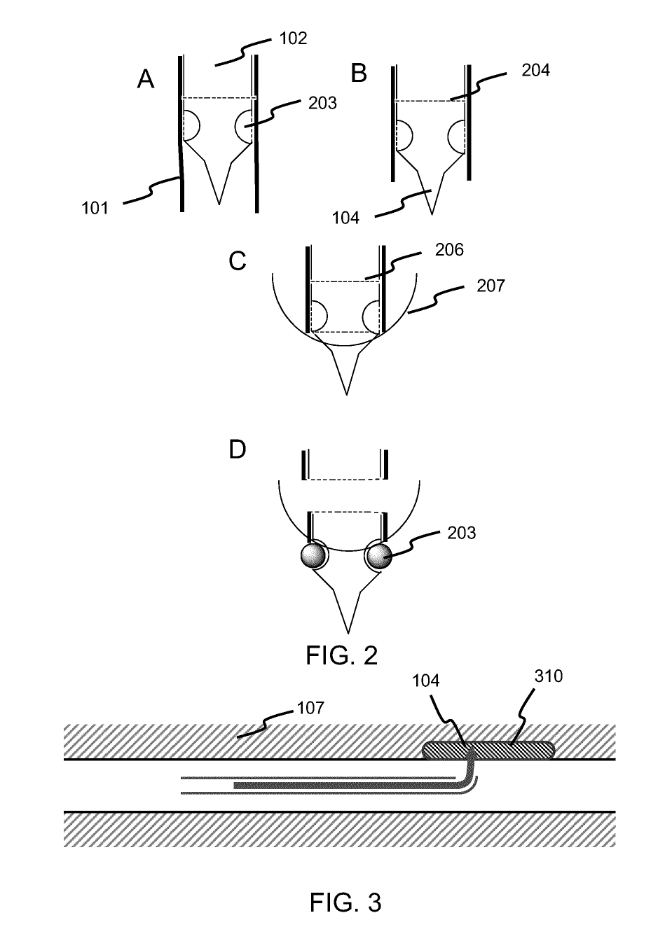

Another embodiment of the catheter device of the present disclosure is illustrated in FIG. 2. Like the catheter in FIG. 1, the catheter device in FIG. 2 has an interior catheter wall (101) defining a lumen in the catheter. Within the lumen is a slideable puncture device 102.

FIG. 2A shows puncture device 102 recessed within the lumen defined by the interior catheter wall (101) and the distal end of the puncture device contains a collar 203 circumscribing the tip 104 and immediately proximal to the collar is a defined breakage or detachment point 204 for the puncture device and optionally for the catheter. FIGS. 2B and 2C show the puncture tip at different stages of protruding from the distal end of the catheter but retaining the collar within the confines of the catheter. FIG. 2D shows that after the collar 203 is allowed to extend beyond the distal end of the catheter, it will expand to form an “O” ring shape. FIG. 2D also shows that upon breakage, the remnants of the puncture stem and puncture tip will cooperatively interact with the “0-ring” to seal the puncture wound.

The collar 203 can be made of any expandable material such as an expandable sponge, a dehydrated hydrogel which will expand upon contact with the fluid of the tissue. Alternatively, a separate lumen leading into the collar can be employed to deliver water to the collar once it passes the vascular wall so as to reduce osmotic shock that may otherwise occur. Yet another form of an expanding collar is an expandable balloon which can be expanded after passing the vascular wall by merely injecting air through a lumen into the balloon.

In one aspect, the puncture tip (104) of the catheter has an expandable collar that enables the catheter to penetrate a vascular wall (FIG. 3) and upon entry into a tissue, the collar extends to seal the vascular wall. The expansion of the collar, in one aspect, is effected by pulling the collar back slightly against the vascular wall.

In some aspects, the puncture device is detachable at a point proximate the puncture tip. Once the puncture tip penetrates a vascular wall and seals it, the catheter and/or the puncture device can be broken at a predetermined detachable point so that the majority of the puncture device and the catheter can be removed from the vascular site. As noted above, the puncture tip can comprise a medicament, which medicament can be released to the tissue once the puncture tip is placed in the tissue behind the vascular wall. In some aspects, the tissue (310) has or is in a diseased or pre-diseased condition requiring medication (FIG. 3). Types of medicaments suitable for delivery to the tissue are described below. In one aspect, the puncture tip is made of a biodegradable material.

In some aspects, there is provided a separate lumen through the puncture device which delivers the medicament preferably as an aqueous solution. The tip can be configured so that the lumen divides at the tip to include multiple micro-channels extending through and outside the tip so as to provide multidirectional flow of medicament. Moreover, if the tip comprises a biodegradable material such as collagen, after delivery of the medicament, the lumen can be flushed with an aqueous solution containing collagenase so as to facilitate the degradation of and closure of the lumen in the tip as necessary. Other combinations useful in the tip include lipids/lipases, cellulose/cellulase, etc.

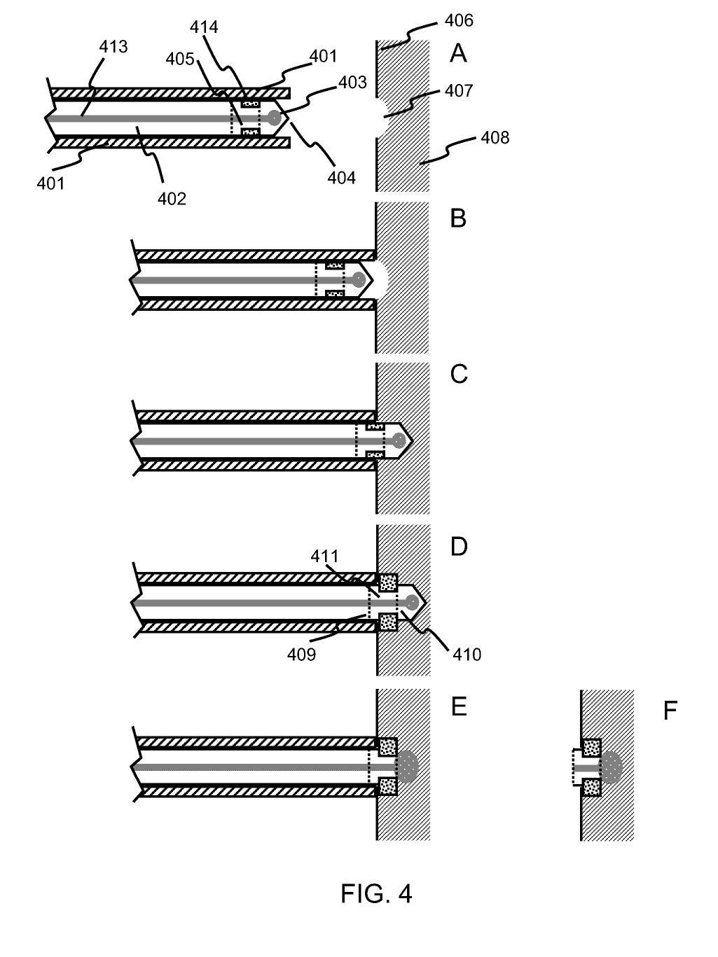

FIG. 4 illustrates yet another embodiment of the catheter device of the present disclosure. The catheter device of FIG. 4 has a catheter wall (401) defining a lumen. The lumen houses the puncture device 413. The puncture device (413) comprises three portions, a head portion (410), a neck portion (411) and a stem portion (409).

The head portion (410) has a sharpened distal end, a puncture tip (404), capable of traversing a vascular wall (406), whether intact of having a rupture or opening (407). The neck portion has a depression (405) on the surface to hold an expandable material (414). In this embodiment, the depression is defined by square walls as opposed to the oval walls of FIG. 2 which illustrates that the particular shape of the depression is not critical.

Moreover, the puncture device has a contour that complements the lumen formed by the interior of the catheter wall, such that the expandable material is held within the depression when the catheter is enclosed in the catheter wall by virtue of the pressure from the contact with the catheter wall (FIG. 4B and 4C). When the expandable material is outside of the catheter wall, however, the expandable material expands to form a collar around the catheter, which collar is useful for sealing a vascular opening (FIG. 4D-E).

In one aspect, the expandable material forms a ring. In another aspect, the expandable material is dispersed, as two or more separate pieces, around the neck portion. For example, when the expandable material contains two pieces, the bottom piece can be impregnated with a coagulate or a glue so as to inhibit bleeding through the puncture site whereas the upper piece can be impregnated with a suitable medicament.

In one aspect, the puncture device comprises a lumen (413) inside thereof. The lumen, in some aspects, has an enlarged end (403) disposed inside the head portion of the catheter. The enlarged end, in on aspect, has a plurality of perforations for releasing a medicament from the lumen. In some aspects, the enlarged end can be further enlarged by pushing air or a liquid material through the lumen into the enlarged end. This is of particular importance as the size of the enlarged end can be designed to complement the collar to form a seal therewith. Alternatively, in those devices of this disclosure which do not employ a collar, the enlarged end can be retracted to fill the puncture wound

In one aspect, the head portion of the puncture device is detachable from the rest of the tip. In one aspect, the head portion is detached from the neck portion of the puncture device by enlarging the enlarged end of the lumen (FIG. 4E). In another aspect, the head portion is detached from the neck portion of the puncture device by a force from the side (FIG. 4F). In one aspect, the head portion is biodegradable.

In some aspects, the neck portion of the puncture device is also detachable from the rest of the tip, such that when at least part of the neck portion enters the vascular wall and the expandable material seals the vascular wall opening, the catheter and stem portion of the puncture device can be removed from the vascular site, leaving the neck portion serving as a seal to the vascular opening (FIG. 4F). Accordingly, in some aspects, the neck portion of the puncture device is made of a biodegradable material.

In some aspects, the enlarged end of the lumen comprises a medicament for delivery to the tissue (408) behind the vascular wall. Examples of medicaments that can be delivered are provided below.

### Catheter Devices with Detachable Puncture Tips

Catheters and puncture devices are also provided, in one embodiment, having the ability to detach at predetermined points. In another embodiment, the catheter has at least one prepositioned detachment mechanisms. The catheter can be any catheter that requires a prepositioned detachment point or points. The detachment mechanism in the catheter is the same as that defined for the puncture device. In another aspect, the catheter has at least two different detachment points with one or more detachment mechanisms. In another aspect, the at least two different detachment mechanisms are selected from (a) detaching by a withdrawing force, (b) detaching by twisting, (c) detaching by dissolving the adhesive used to connect the parts, or detaching with the same type of mechanisms but with different amount of force, different direction of force, or different types of glue or solvent. In one aspect, the different mechanisms comprise different strengths or directions of force or are selected from the group consisting of (a) a protuberance detachably engaged in a complementary recess, (b) a joint detachable by a withdrawing force, (c) a joint detachable by a twisting force and (d) a joint with glue detachable by a solvent. Various types of detachment mechanisms have been described above and others will be further provided as follow.

It is understood that these detachment mechanisms are complementary to any of those known in the art and can be used in a catheter, in a puncture device and in combinations thereof.

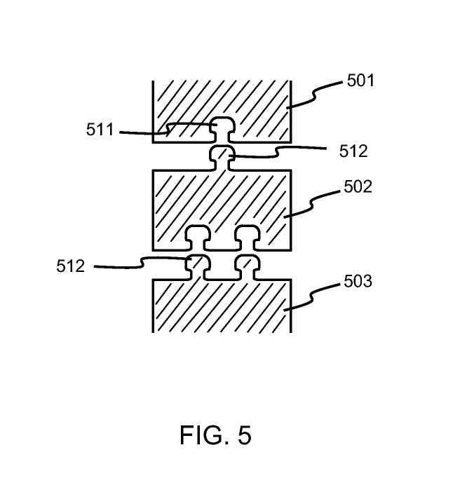

In one embodiment as illustrated in FIG. 5, the at least two different detachment mechanisms can be made by combining parts of the puncture device in such a manner that a predetermined backward force would be capable of causing detachment. In FIG. 5, distal end 501 of the puncture device is mated with the proximal end 502 through a protuberance 507 and recess 511. In this embodiment, protuberance 507 is deformable under a defined backward pressure such that under such pressure, the distal and proximal ends will separate. Further, in another aspect, a second optional mating site is provided to ensure that if the first site becomes inaccessible due to incorporation, e.g., into the embolic mass, the second site can be used for separation.

In such an embodiment, the backward pressure required to separate the second site is engineered to be greater than the first site so that separation can be controlled. Alternatively, the second site can have an orthogonal means for separation from the first site. For example, the second site can be a weakened portion of puncture device which will detach upon continued twisting. That is to say that if the puncture tip and the first detachment site are locked into place by e.g., an embolic mass, then that portion of the puncture device is locked such that twisting of the puncture site from the proximal end will induce stress on the second site resulting in detachment.

In another embodiment, metal bands (not shown) can be included at different points in the puncture device so as to permit the clinician to ascertain where the device has separated.

Alternatively, targeted separation can be achieved by use of a glue or other adhering mechanism having a defined degree of adhesiveness so that the force required to separate at the desired site can be readily ascertained. When multiple sites for separation are desired, then one only need to use glues of differing adhesiveness.

### Medicaments

In various embodiments of the catheter devices of the present disclosure, the catheter, the puncture tip, or a lumen in the catheter of the tip is loaded with a medicament for delivery to a tissue at a vascular site. The medicament can be useful for treating a disease or condition at the vascular site, or facilitating healing of a rupture.

In one aspect, the medicament comprises an anti-inflammatory agent. Non-limiting examples of anti-inflammatory agents include steroids such as glucocorticoids and non-steroidal anti-inflammatory drugs (NSAID) including ibuprofen, fenoprofen, aspirin, mefenamic acid, nimesulide and licofelone, and combinations thereof

In another aspect, the medicament comprises a thrombotic agent, such as but not limited to, zeolites, thrombin glue, fibrin glue, desmopressin, a coagulation factor concentrate, tranexamic acid, aminocaproic acid and aprotinin.

In yet another aspect, the medicament comprises a pain reliever. Commercially available pain relievers include, for example, Tylenol®, Advil®, Aleve®, Mortin®, and Excedrin®.

In still another aspect, the medicament comprises an anti-cancer agent, such as but not limited to, nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors, and hormonal agents, inter alia.

In one aspect, the anti-cancer agent is a small molecule drug such as Actinomycin-D, Alkeran, Ara-C, Anastrozole, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, and Xeloda.

In another aspect, the catheter devices of the present disclosure are suitable for delivering a biological anti-cancer agent, examples of which include, Herceptin, Rituximab Asparaginase, Cetuximab, Brentuximab vedotin, Canakinumab, Denosumab, Gemtuzumab, Ibritumomab tiuxetan, Muromonab-CD3, Ofatumumab, Panitumumab, Tositumomab, and Trastuzumab.

In a particular aspect, the catheter of the present disclosure can be used to deliver an anti-cancer agent to the brain for treating a brain tumor. Selection of anti-cancer agents for brain tumors patient depends on several factors, including the patient's age, Karnofsky Score and any previous therapy the patient has received. At www.neurooncology.ucla.edu/Performance/GlioblastomaMultiforme.aspx, The University of California at Los Angeles has published a list of anti-neoplastic agents that are suitable for treating brain tumors, which list is reproduced in Table 1 below.

TABLE 1
Known anti-neoplastic agents for treating brain tumors

| | | |
|---|---|---|
| 5FC | Accutane Hoffmann-La Roche | AEE788 Novartis |
| AMG-102 | Anti Neoplaston | AQ4N (Banoxantrone) |
| AVANDIA (Rosiglitazone | Avastin (Bevacizumab) Genetech | BCNU |
| Maleate) | | |
| BiCNU Carmustine | Carboplatin | CCI-779 |
| CCNU | CCNU Lomustine | Celecoxib (Systemic) |
| Chloroquine | Cilengitide (EMD 121974) | Cisplatin |
| CPT -11 (CAMPTOSAR, | Cytoxan | Dasatinib (BMS-354825, |
| Irinotecan) | | Sprycel) |
| Dendritic Cell Therapy | Etoposide (Eposin, Etopophos, | GDC-0449 |
| | Vepesid) | |
| Gleevec (imatinib mesylate) | GLIADEL Wafer | Hydroxychloroquine |
| Hydroxyurea | IL-13 | IMC-3G3 |
| Immune Therapy | Iressa (ZD-1839) | Lapatinib (GW572016) |
| Methotrexate for Cancer | Novocure | OSI-774 |
| (Systemic) | | |
| PCV | Procarbazine | RAD001 Novartis (mTOR |
| | | inhibitor) |
| Rapamycin (Rapamune, | RMP-7 | RTA 744 |
| Sirolimus) | | |
| Simvastatin | Sirolimus | Sorafenib |
| SU-101 | SU5416 Sugen | Sulfasalazine (Azulfidine) |
| Sutent (Pfizer) | Tamoxifen | TARCEVA (erlotinib HCl) |
| Taxol | TEMODAR Schering-Plough | TGF-B Anti-Sense |
| Thalomid (thalidomide) | Topotecan (Systemic) | VEGF Trap |
| VEGF-Trap | Vincristine | Vorinostat (SAHA) |
| XL 765 | XL184 | XL765 |
| Zarnestra (tipifarnib) | ZOCOR (simvastatin) | |

In some aspects, the medicament comprises one, or two, or three, or four, or five or more agents selected from any of the above or their combinations.

### Methods

Methods of using the catheter devices and tips are also provided. Some methods are apparent from the description of the devices and as illustrated in, for instance, FIGS. 1, 3 and 4.

In general, the present disclosure provides methods for penetrating a vascular wall for the purpose of sealing an opening on the vascular wall and/or delivering a medicament to the tissue around the penetrating site. The methods, in some aspects, entails placing a catheter to the vascular site, extending a puncture tip from the catheter to traverse through the vascular wall. Upon entry into the tissue behind the vascular wall, the catheter seals the opening.

Further, the catheter can comprise medicament or be accompanied by a lumen or another tip for delivering a medicament to the tissue at the vascular site. Results achieved by the drug delivery are apparent from the type of the medicaments. For instance, the method can be useful in treating information that occurs during a stroke, sealing an aneurysmal rupture, relieving a pain, or treating cancer.

In a particular embodiment, it is contemplated that the catheter devices of the present disclosure are useful in treating a condition in the brain, such as stroke, aneurysmal rupture and brain cancer. Accordingly, one aspect of the disclosure provides a methods for treating a brain cancer. The advantage of such a method can be readily appreciated by the skilled artisan, as there is a constant need for a feasible strategy to deliver a drug through the blood brain barrier (BBB). Using a catheter of the present disclosure, in this respect, directly delivers a drug through the BBB thereby overcoming such a difficulty.

Further, as portions of the catheters can be detachable, biocompatible and biodegradable, the present disclosure provides methods for sealing vascular openings and delivering a medicament safely.

Additionally, in one embodiment, the present disclosure provides a method for sealing an opening at a vascular site, which method comprises:

placing a catheter at the vascular site,
wherein the catheter comprises a proximal end and a distal end and has at least one lumen traversing from the proximal end to the distal end,
wherein the lumen comprises an extendible puncture device which comprises a puncture tip at its distal, a neck portion and a stem at its proximal end, and
wherein the neck portion of the puncture device comprises a collar which, upon entry through the puncture opening, expands to form a seal around the opening; and
extending the puncture device so as to extend the puncture tip and neck through the vascular wall such that the collar expands thereby sealing the opening.

In one aspect, the vascular site has an aneurysm. In another aspect, the puncture device is capable of delivering a medicament to the vascular site, and the method, accordingly, can include delivering the medicament to the vascular site.

In another aspect, the method further comprises detaching a distal end of the puncture device so as to remove the catheter and the portion of the puncture device proximal to the catheter from the vascular site. In certain aspects, the distal end of the puncture device is biodegradable.

In another embodiment, the catheter has lumen that can be used to deliver an embolic composition to a rupture of an aneurysm. This is illustrated in FIG. 1E, in which the puncture tip of the catheter can deliver a medicament to the tissue behind the vascular wall and the lumen of the catheter can be used to deliver an embolic agent to fill the balloon.

Accordingly, one embodiment of the present disclosure provides a method for treating a hemorrhagic stroke in a patient due to rupture of an aneurysm, which method comprises: inserting a first catheter into the patient such that its distal end is proximate to or in an aneurysmal sac which catheter comprises a lumen having a puncture device slideably engaged therein said device having a puncture tip at its distal end and a stem at the proximal end and further wherein the puncture tip or the region proximate thereto is capable of delivering a medicament to the site punctured by the tip;

inserting a second catheter into the patient such that its distal end is proximate to or in the the aneurysmal sac wherein the catheter comprises a proximal end and a distal end and has at least one lumen traversing from the proximal to the distal end wherein the lumen is sized to deliver an embolic composition or material to the vascular site

filling the aneurysmal sac with an embolic composition or material so as to arrest blood flow into the aneurysm;

puncturing the vascular wall with the puncture tip of the puncture device; and delivering medicament to the site punctured by the puncture tip.

An “embolic agent” causes occlusion of blood vessels by introducing emboli at a vascular site. Non-limiting examples include liquid embolic agents such as n-butyl-2-cyanoacrylate and ethiodol; sclerosing agents such as ethanol, ethanolamine oleate, and sotradecol; particulate embolic agents such as gelfoam, polyvinyl alcohol (PVA) and acrylic gelation microspheres; and mechanical occlusion devices such as coils and balloons as well as ONYX®. Suitable medicaments are described above.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A catheter comprising a proximal end, a distal end, and at least two lumen traversing from the proximal to the distal ends, wherein the first lumen is sized to deliver an embolic composition or device to a vascular site, and the second lumen comprises a puncture device slideably engageable within said lumen, wherein said puncture device comprises a puncture tip capable of penetrating the vascular wall at the vascular site and capable of delivering a medicament to the vascular site punctured by said puncture tip.

2. A catheter comprising a proximal end, a distal end, and a lumen traversing from the proximal to the distal ends, wherein the lumen comprises a puncture device slideably engageable within said lumen, wherein said puncture device comprises a puncture tip comprising a closed collar which, after puncturing a vascular wall, flattens to form a surface proximate the punctured vascular wall to seal the punctured vascular wall.

3. A catheter comprising a proximal end, a distal end, and a lumen traversing from the proximal to the distal ends, wherein the lumen comprises a puncture device slideably engageable within said lumen, wherein said puncture device comprises a puncture tip comprising an expandable collar circumscribing said puncture tip which, after puncturing a vascular wall, expands to form a ring proximate the punctured vascular wall to seal the punctured vascular wall.

4. The catheter of claim 2, wherein the puncture tip comprises one or more perforations to deliver a medicament.

5. The catheter of claim 2, wherein the puncture device comprises one or more detachment points.

6. A catheter comprising a proximal end, a distal end, and a lumen traversing from the proximal to the distal ends, wherein the lumen comprises a puncture device slideably engageable within said lumen, wherein said puncture device comprises a puncture tip and at least two detachable points proximate said puncture tip, wherein said at least two detachment points are configured to be broken with different mechanisms.

7. The catheter of claim 6, wherein the different mechanisms comprise different strengths or directions of force or are selected from the group consisting of (a) a protuberance detachably engaged in a complementary recess, (b) a joint detachable by a withdrawing force, (c) a joint detachable by a twisting force and (d) a joint with glue detachable by a solvent.

8. A detachable catheter comprising a predetermined detachment point and a detachment mechanism selected from (a) detaching by a withdrawing force, (b) detaching by twisting, (c) detaching by dissolving the adhesive used to connect the parts, or detaching with the same type of mechanisms but with different amount of force, different direction of force, or different types of glue or solvent.

9. A detachable catheter comprising at least two predetermined detachment points having different detachment mechanisms are selected from (a) detaching by a withdrawing force, (b) detaching by twisting, (c) detaching by dissolving the adhesive used to connect the parts, or detaching with the same type of mechanisms but with different amount of force, different direction of force, or different types of glue or solvent.

10. A detachable catheter comprising a first proximal section and a second distal section wherein the proximal section comprises mating means with the distal section such that the two sections when mated form a integrated catheter and further wherein the mating sections can be detached by unmating the proximal and distal sections.
